(19) United States
(12) Patent Application Publication
(10) Pub. No.: US 2014/0073743 A1
(43) Pub. Date: Mar. 13, 2014

(54) POLY(OCTYLCYANOACRYLATE)-POLYISOBUTYLENE POLYMER CONETWORK, METHOD FOR THE PRODUCTION THEREOF AND USES THEREOF

(71) Applicants: Joseph Kennedy, Akron, OH (US); Gabor Erdodi, Stow, OH (US); Ryan Gasser, Norton, OH (US); Juay Seng Tang, Singapore (SG)

(72) Inventors: Joseph Kennedy, Akron, OH (US); Gabor Erdodi, Stow, OH (US); Ryan Gasser, Norton, OH (US); Juay Seng Tang, Singapore (SG)

(73) Assignee: THE UNIVERSITY OF AKRON, Akron, OH (US)

(21) Appl. No.: 13/984,367

(22) PCT Filed: Feb. 7, 2012
(86) PCT No.: PCT/US2012/024060

§ 371 Date: Nov. 21, 2013

Related U.S. Application Data
(60) Provisional application No. 61/441,813, filed on Feb. 11, 2011.
(60) Provisional application No. 61/559,778, filed on Nov. 15, 2011.

(51) Int. Cl. A61L 24/06

(52) U.S. Cl. CPC A61L 24/06
USPC 525/295

(57) ABSTRACT

A polymer conetwork formed from the polymerization reaction of octyl-cyanoacrylate and a tri-telechelic star polymer comprising polyisobutylene terminated with cyanoacrylate groups (Ø(PIB-CA)₃), wherein the ratio of octyl cyanoacrylate to Ø(PIB-CA)₃ is from about 10:1 to about 40:1.

### CROSS REFERENCE TO RELATED APPLICATIONS

This application claims the benefit of U.S. Provisional Patent Application No. 61/441,813, filed Feb. 11, 2011 and claims the benefit of U.S. Provisional Patent Application No. 61/559,778, filed Nov. 15, 2011, both of which are hereby incorporated by reference.

### TECHNICAL FIELD

This invention relates to a polyisobutylene-based conetwork and, more particularly, to a poly(octyl cyanoacrylate)-polyisobutylene conetwork. In addition to the polymer conetwork, a method of producing the polymer conetwork is provided, as well as a number of uses for the polymer conetwork are disclosed.

### BACKGROUND OF THE INVENTION

There is a great need in biomedical applications, including orthopedic practice, for sealants of wounds and surgical cuts. Such sealants contemplated could range from sealants used for wound healing and wound closure on the skin to sealants used to permanently seal scalpel cuts and puncture wounds made by large bore injection needles in the course of various procedures.

For example, iatrogenic defects made in the annulus fibrosa during discectomies do not heal. Rather, the compromised disc never effectively heals and the nucleus pulposus may reherniate through the unrepaired defect. Furthermore, the defect in the annulus accelerates disc degeneration and is a source of pain. Iatrogenic annular defects are made in various procedures, including discography, intradiscal electrothermy, and emerging nuclear replacement/regenerative technologies.

At the present no satisfactory orthopedic sealant is known that could be used to satisfy this need. The intervertebral disc subsequently undergoes accelerated degeneration and the patient requires a spinal fusion some years later. Some implants have been proposed to resolve the issue but these were introduced without biomechanical considerations. Mechanical barriers have been recently proposed but are fundamentally different from an annulus sealant in that it (1) lack the ability to reconstruct the annulus directly and restore motion, (2) cannot prevent the leakage of smaller particles from within the nucleus pulposus, (3) are more technically difficult to employ, and (4) would carry a significant risk of neurologic injury if extruded into the canal. No long term data is available on these products.

Stated differently, an unmet clinical need of the repair of torn annulus following an episode of lumbar disc herniation remains a problem today. Lumbar disc herniation, common in healthy adults, causes excruciating pain and immobility. Surgical treatment is usually performed in patients with intractable pain after failure of at least six weeks of conservative treatment. The current clinical “gold standard” to treat disc herniation is lumbar discectomy, a surgical procedure to remove part of the intervertebral disc. Discectomy, however, results in loss of disc height and in the long term is associated with intraforaminal compression, and also recurrent back and leg pain. The more rapid disc space narrowing places more stress on the cartilage endplates, annulus fibrosus and posterior facet joints and appears to result in accelerated disc degeneration and a poorer clinical outcome. Less invasive endoscopic and microsurgical techniques have certain advantages over open discectomy and are alternative clinical procedures. A high incidence of disc herniation recurrence, at a rate of up to 18%, is associated with these less invasive techniques, which involve the removal of sequestrated disc material without discectomy. The damaged annulus fibrosus following an episode of disc herniation is unable to repair itself. The remaining nucleus pulposus could thus again extrude under excessive load through the damaged annulus fibrosus, and result in recurrent disc herniation. Discectomy and less invasive endoscopic and microsurgical techniques continue to be the current surgical solutions of choice to treat lumbar disc herniation.

Homopolymers of alkyl (methyl-, ethyl-, butyl-, octyl, etc.) cyanoacrylates are well known to the art, and their use as adhesives (e.g., glues) has been thoroughly investigated. For example, Superglue® is essentially ethyl-cyanoacrylate (Et-CA), while and Dermabond® is octyl-cyanoacrylate (Oct-CA). It is well known that these monomers readily polymerize upon exposure to traces of moisture on surfaces.

Most of these polymers having low molecular weight, namely, alkyl cyanoacrylates (methyl-, ethyl-, and butyl-), cannot be used as sealants inside the body because of the toxicity of unprotected or exposed —CN groups in these molecules. The toxicity of these materials decreases with increasing molecular weight of the alkyl group, and, indeed, octyl cyanoacrylate, Oct-CA, is an FDA approved tissue adhesive (Dermabond®). However, going back to the repair of the annulus issue, direct repair of the annulus using sutures was studied biomechanically in a sheep model with internal pressurization of the disc, and sutures were found to have no significant repair effect. In a study to investigate the combined use of suture and 2-octyl cyanoacrylate for intervertebral disc repair, it was found to sustain only 16,900 loading cycles which was lower than the desired 100,000 cycles. In that same study, sutures alone sustained 3400 loading cycles and fibrin glue with suture sustained 8600 loading cycles. The study, conducted in 2008, provided biomechanical evidence to surgeons that repair of tears in the annulus with suture alone or combined with 2-octyl cyanoacrylate was not a viable solution to prevent recurrence of disc herniation.

Multi-arm polyisobutylene stars are well known in the art and have been developed and patented by at least one of the named inventors. The production of such polyisobutylenes provide for a core (Ø) with a desired number of polyisobutylene arms extending therefrom.

There are many potential biomedical applications with polyisobutylene with attachment of various polymers at the end of each arm. One clinical example where polyisobutylene has been adopted is poly(styrene-b-isobutylene-b-styrene), which is currently used as a coating in the Taxus® Drug Eluting Stent. Another potential application is for all applications where octyl cyanoacrylate (Dermabond®) is currently employed and more flexibility is required.

To that end, cyanoacrylate-telechelic three-arm star polysiobutylene should be prepared. Cyanoacrylate-telechelic three-arm star polysiobutylene, Ø(PIB-CA)₃, was first prepared in 1991. A low viscosity syringible and injectible Ø(PIB-CA)₃ was subsequently developed in 2007. It was found that a bolus of covalently linked PIB rubber “superglue” was created when Ø(PIB-CA)₃ was injected into (egg) protein and the properties could be controlled by addition of polyethyl-2-cyanoacrylate (Et-CA). On its own, Ø(PIB-CA)₃ has a tensile strength of 1.6 MPa, Young's Modulus of 4.9 MPa, and an elongation of 70%. Comparatively, the tensile strength of clinically available 2-octyl cyanoacrylate based “superglue”, Dermabond® (Ethicon, J&J) and SurgiSeal™ (Adhezion Biomedical), is less than 0.1 MPa.

Furthermore, it is known that high molecular weight cyanoacrylacrylate-ended tri-telechelic polyisobutylene Ø(PIB-CA)₃ (Mn ˜2500 g/mol) are nontoxic in rats in vivo. Without being bound by theory, it is believed that the biocompatible high barrier rubbery PIB moiety effectively envelops and shields the noxious cyanoacrylate groups from the surrounding tissue and the permanently sequestered -CA groups are rendered harmless.

Thus, the need exists for a polymer conetwork that provides the flexibility, elongation and tensile strength of Ø(PIB-CA)₃ with the “superglue” properties of certain alkyl cyanoacrylates, particularly, octyl cyanoacyrlates. The polymerization of simple alkyl cyanoacrylates, and specifically, octyl cyanoacrylates, with Ø(PIB-CA)₃ to provide useful conetworks is believed to be particularly desirable in a number of biomedical applications.

### SUMMARY OF INVENTION

Therefore, it is an aspect of the present invention to provide a polymer conetwork of poly(octyl cyanoacrylate) and tri-telechelic, cyanoacrylate-ended polyisobutylene.

Another aspect of the present invention is to provide a method for the production of this polymer conetwork.

A further aspect of the present invention is to provide a polymer conetwork that may be used for wound closure.

A further aspect of the present invention is to provide a polymer conetwork that may be used for sealing surgical cuts and puncture wounds.

A further aspect of the present invention is to provide a polymer conetwork that may be used as an orthopedic sealant.

A further aspect of the present invention is to provide a polymer conetwork that may be used for repair of torn annulus.

Other aspects of the present invention may be found in one or more of the various biomedical applications for which the polymer conetwork could prove or has proven to be useful.

Any one or more of the foregoing aspects, together with the advantages thereof over the known art relating to polymer conetworks, which shall become apparent from the specification that follows, are accomplished by the invention as hereinafter described and claimed.

In general, the present invention provides a polymer conetwork formed from the polymerization reaction of octyl-cyanoacrylate and a tri-telechelic star polymer comprising polyisobutylene terminated with cyanoacrylate groups (Ø(PIB-CA)₃), wherein the ratio of octyl cyanoacrylate to Ø(PIB-CA)₃ is from about 10:1 to about 40:1. The polymerization reaction is initiated by nucleophlic groups located on the surface to be covered by the polymer conetwork, such as, in one embodiment, skin. The polymer conetwork exhibits higher elongation than a homopolymer of octyl cyanoacrylate and exhibits strength sufficient to hold two pieces of skin together (at least 5N).

### BRIEF DESCRIPTION OF THE DRAWINGS

FIG. 1 is a representative sketch of the structures of the initiating skin or tissue surface carrying nucleophilic groups, alkyl cyanoacrylate (R-CA) and tri-telechelic cyanoacrylate polyisobutylene (Ø(PIB-CA)₃);

FIG. 2 is a representative sketch of the initiation of the poly(cyanoacrylate) chain; and

FIG. 3 is a representative sketch of a polymer conetwork of poly(alkyl cyanoacrylate) and polyisobutylene, wherein the poly(alkyl cyanoacrylate) is covalently attached to a surface.

### DETAILED DESCRIPTION OF THE INVENTION

As noted hereinabove, the present invention seeks to provide a novel polymer conetwork composition of matter suitable for any of a number of biomedical applications, from wound closure and healing of skin tissue, to sealant for surgical cuts, to a sealant for surgical cuts and punctures to treat lumbar disc herniation. The composition is a conetwork comprising a copolymer of 2-n-octyl-cyanoacrylate (Oct-CA) and cyanoacrylate-terminated tri-telechelic polyisobutylene (Ø(PIB-CA)₃). The polymerization of Oct-CA with Ø(PIB-CA)₃ is rapidly initiated by the skin, blood or other living (or dead) tissue when a mixture of the liquid starting materials is sprayed, coated or otherwise applied over wounds or surgical cuts. In one embodiment, the composition may be a sprayed coating or film that rapidly solidifies into a robust rubbery protecting barrier. The tissue, e.g., skin, or more accurately, the nucleophilic groups (—OH, NH2, etc.) on the surface of the skin, is, in effect, the “catalyst” of the polymerization, i.e., the agent that initiates the polymerization. The polymer that forms is a biocompatible biostable hydrophobic elastomeric barrier to bacterial invasion that keeps the coated skin moist, thereby promoting healing. The barrier, because of the specific catalyzed initiation mechanism, adheres strongly by covalent bonds onto the surface of the tissue. Because of the absence of Ø(PIB-CA)₃ in earlier purely polyalkyl-cyanoacrylate wound closures, earlier wound closures did not exhibit such advantageous combination of elastomeric properties.

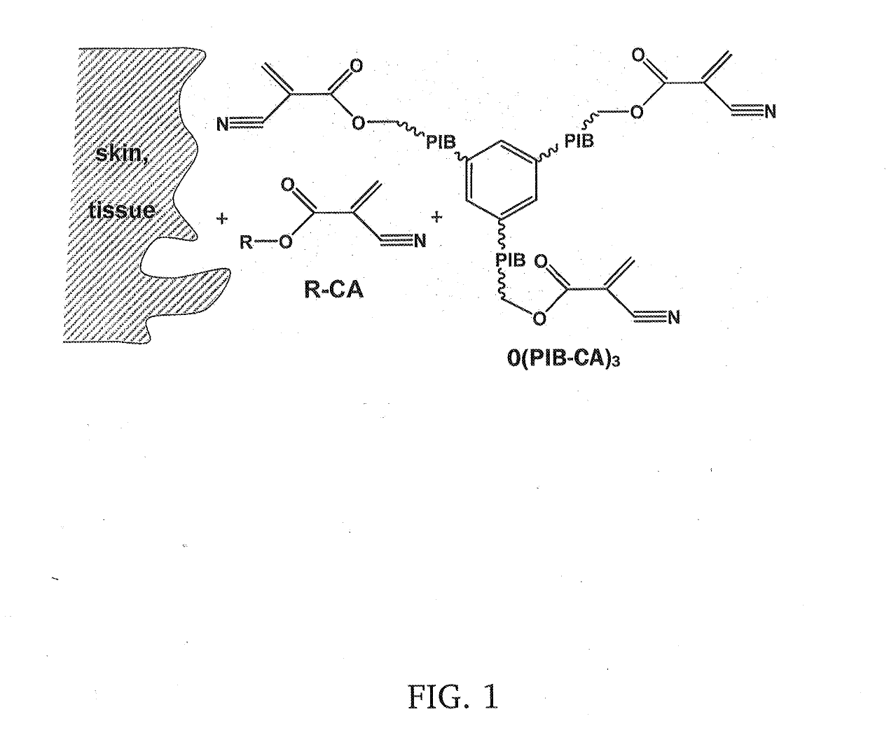

To understand structurally the polymerization mechanism, reference is made to the drawings. FIG. 1 is a sketch of the initiating structures set forth in the application. Specifically, the chemical structure of alkyl cyanoacrylate is set forth as R-CA. It will be appreciated for at least one embodiment of this invention that R is an octyl group (hereinafter referred to as Oct-CA). The other starting material is cyanoacrylate-terminated tri-telechelic polyisobutylene, denoted as Ø(PIB-CA)₃, whit the structure shown in FIG. 1.

Because both Ø(PIB-CA)₃ and Oct-CA contain polymerizable cyanoacrylate (CA) groups they can readily produce polymers. Polymer (i.e., conetwork or polymer conetwork) composition can be controlled by using desired amounts of the two ingredients. Overall, the polymer conetwork composition will reflect the relative composition of the starting monomers.

Because both monomers, Ø(PIB-CA)₃ and Oct-CA, are liquids, their mixtures can be delivered in a number of different ways. In one embodiment, the liquids are delivered as described above as spraying to provide a suitable coating or film of the composition onto the desired tissue. In another embodiment, the liquids may be delivered by syringe, injecting the composition to a suitable site. By allowing such monomer mixtures to polymerize in situ, solid rubbery plugs can form exactly where the mixture was injected, i.e., where the seal is needed. The fact that Ø(PIB-CA)₃, having a Mn approximately=3000 g/mol, and Oct-CA are miscible was surprising because the very similar cyanoacrylates Methyl-, Ethyl-, and Butyl-CA are completely immiscible with Ø(PIB-CA)₃, and when such binary systems are mixed they do not form a homogeneous phase but remain separate.

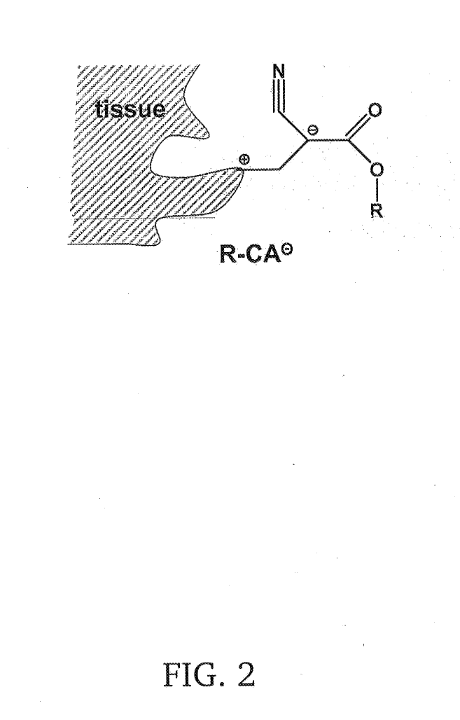

Most desirably, and in one embodiment, polymerization of Ø(PIB-CA)₃, plus Oct-CA mixtures occur spontaneously in bulk (i.e., absence of solvent) upon contact with living tissue. Initiation of polymerization does not require the addition of an extra catalyst because living tissues contain an abundance of nucleophilic groups (e.g., —NH2, —OH) which rapidly initiate polymerization at their surfaces. As shown in FIG. 2 and the following equation, the initiation reaction that takes place at the surface between nucleophilic groups on the tissue surface, e.g., on the skin, and the cyanoacrylates:

T-NH₂+CH₂═C(CN)COOR→T-NH₂⁺—CH₂—C(CN)—COOR

where T=tissue, and R=Octyl or PIB.

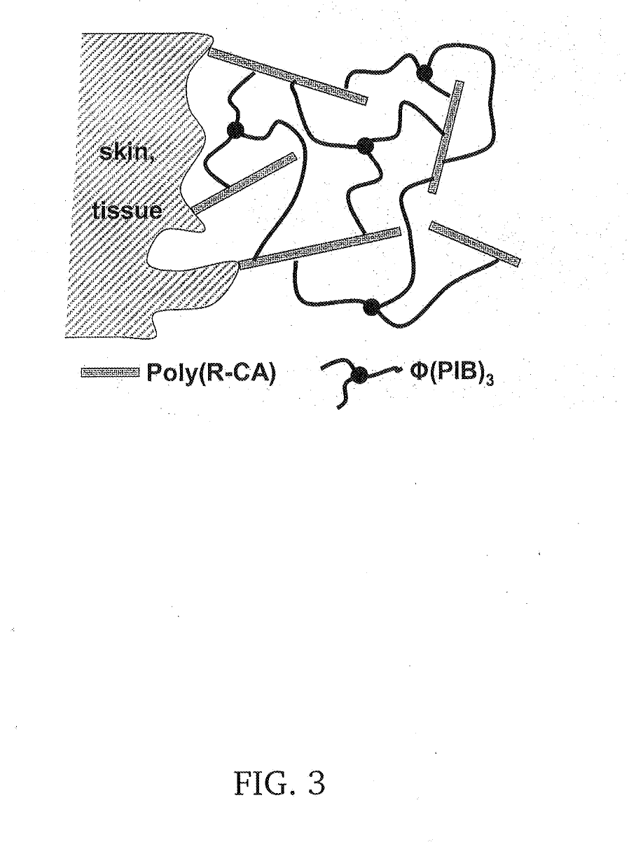

As indicated by the equation and in FIG. 2, initiation produces a strong covalent chemical bond between the living tissue (T) and the CA group. In other words chemical linkages arise between the tissue and the conetwork. Because the molar concentration of CA groups is much higher in the relatively low molecular weight Oct-CA than in the high molecular weight Ø(PIB-CA)₃, the chances of surface initiation with Oct-CA is much higher than that with Ø(PIB-CA)₃ (see FIG. 3). The formation of a permanent chemical bond between the conetwork and living tissue is very highly desirable.

It will be appreciated that polymerization occurs with each CA group attaching either to the tissue (generally where the CA group is a part of the Oct-CA) or to another of CA group, whether that CA group is from Oct-CA or from Ø(PIB-CA)₃. Thus, the two polymers PIB and poly(Oct-CA) are covalently linked to each other, and the trifunctional Ø(PIB-CA)₃ acts as a crosslinker of the conetwork (see FIG. 3).

The result polymer conetwork of poly(Oct-CA)-PIB has been found to contain segregated nonpolar PIB and polar Oct-CA phases. The Tg of the PIB phase is ˜−70° C. and that of the poly(Oct-CA) is ˜40° C., i.e., the difference in Tg's between the two phases is ˜110° C.

Perhaps more importantly, the mechanical and chemical properties (e.g., hardness, toughness, elasticity, elongation, strength etc.) of the rubbery polymer conetwork of poly(Oct-CA)-PIB can be engineered and/or controlled relative composition of the two starting ingredients and dictating or controlling the molecular weight of the Oct-CA and Ø(PIB-CA)₃ and/or dictating or controlling the ratio or relative concentration of the two starting materials. It is believed to be advantageous to prepare conetworks whose mechanical properties are similar to the living tissue where the composition will be situated.

For example, the rubbery character (stretchiness) of the conetwork can be increased by increasing the concentration of the Ø(PIB-CA)₃ in the polymer conetwork. Other properties can be controlled by controlling the molecular weights (Mn) of the co-network segments (i.e., the number average molecular weights of the poly(O-CA) and Ø(PIB-CA)₃, respectively). While any molecular weight range can be used, one suitable range would be to provide the molecular weight of poly(O-CA) as from about 3000 g/mol to 5000 g/mol, with about 4000 g/mol being suitable for one embodiment, and that of Ø(PIB-CA)₃ being from about 2000 g/mol to about 4000 g/mol, with about 3000 g/mol being suitable in one embodiment. Thus, it will be appreciated that, for the three arm tri-telechelic cyanoacrylate PIBs, each PIB arm is about 1000 g/mol. The production of these two starting molecules is well known to those of skill in the art.

One drawback to the production of the polymer conetwork of the present invention is that, due to its high number average molecular weight relative to the homopolymer, poly(Oct-CA), as available under the brand name Dermabond® (Ethicon, J&J), the rate of polymerization of the conetwork is slower and the polymer cures slower than the poly(Oct-CA) homopolymer. Thus, while polymerization will occur regardless of the ratio of Oct-CA groups to Ø(PIB-CA)₃, the rate is only a problem with where molar concentrations of Oct-CA are too low. Accordingly, in one embodiment, the ratio of poly(Oct-CA) to Ø(PIB-CA)₃ is from about 10:1 to about 40:1. In other embodiments, the ratio is from about 15:1 to 35:1 and in still other embodiments, the ratio is from about 20:1 to about 30:1. A ratio of greater than 10:1 should provide a sufficient molar concentration of Oct-CA to provide an adequate rate of polymerization. In a further embodiment, the ratio is greater than 20:1.

In order to demonstrate practice of the invention, various embodiments of the conetwork were prepared and tested. In a first embodiment, miscibility and in vitro pressuration tests were performed on compositions prepared as follows.

Oct-CA (Dermabond®) was purchased from eSutures.com. The synthesis and characterization of Ø(PIB-CA)₃, Et₂N-PIB-NEt₂, and HO-PIB-OH are well known and have been described in the prior art. The molecular weights of the Ø(PIB-CA)₃, Et₂N-PIB-NEt₂ and HO-PIB-OH were 2500, 3500 and 2000 g/mol, respectively as determined by GPC.

To determine the miscibility of the various alkyl cyanoacrylates with Ø(PIB-CA)₃ freshly prepared Ø(PIB-CA)₃ was mixed with ethyl cyanoacrylate (Et-CA), butyl cyanoacrylate (Bu-CA) and octyl cyanoacrylate (Oct-CA) under a blanket of gaseous N₂ and transferred to disposable polypropylene syringes equipped with a 18 gauge needle. TABLE 1 shows the ingredients used, the compositions of mixtures, and miscibilities determined by visual observation.

TABLE 1
Ingredients, Charges Miscibilities, of Mixtures

| | 0[PIB-CA]₃ | HO-PIB-OH | Et₂N-PIB-NEt₂ | Comonomer | | |
|---|---|---|---|---|---|---|
| Samp | % | % | % | type | % | Miscibility* |
| 1 | 0 | 0 | 0 | Et-CA | 100 | Immiscible |
| 2 | 90 | 0 | 0 | Et-CA | 10 | Immiscible |
| 3 | 70 | 0 | 0 | Et-CA | 30 | Immiscible |
| 4 | 70 | 0 | 0 | Bu-CA | 30 | Immiscible |
| 5 | 85 | 0 | 0 | Bu-CA | 15 | Immiscible |
| 6 | 0 | 0 | 0 | Oct-CA | 100 | Miscible |
| 7 | 40 | 20 | 0 | Oct-CA | 40 | Miscible |
| 8 | 40 | 0 | 20 | Oct-CA | 40 | Miscible |
| 9 | 60 | 0 | 0 | Oct-CA | 40 | Miscible |
| 10 | 90 | 0 | 0 | Oct-CA | 10 | Miscible |

*by visual observation

Repeated attempts to prepare poly(Et-CA)-PIB and poly(Bu-CA)-PIB conetworks by bulk polymerization failed because of the incompatibility of the ingredients. These mixtures gave heterogeneous pastes, instead of the desired strong rubbery conetworks. With these mixtures conetworks can be prepared only by the use of a common organic solvent, e.g., THF, which dissolves both ingredients. However, this solution method is unsuitable for the preparation of conetworks for medical applications because of the presence of the toxic organic solvent.

Experiments were carried out by the use of poly(Oct-CA)-PIB mixtures in the presence of a H₂N-PIB-NH₂ (Mn=3000 g/mol) macroinitiator. However, conetworks failed to form even in the presence of this strong nucleophile due to immiscibility.

Undeterred by the results obtained with Et-CA and Bu-CA, polymerizations were also carried out with Oct-CA. In these experiments, various proportions of Oct-CA and Ø(PIB-CA)₃ were mixed in the bulk and were exposed in the absence of solvents to the nucleophiles, HO-PIB-OH and NH₂-PIB-NH₂ (see entries 7-8 in TABLE 1). It was found that these experiments yielded strong rubbery networks.

Subsequently, experiments were carried out by contacting homogeneous Oct-CA plus Ø(PIB-CA)₃ mixtures with bovine vertebrates. These experiments were carried out as follows.

Lumbar spinal columns of 20 weeks old bovine were procured locally from an abattoir. They were vacuum sealed in plastic bags and stored in −20° C. freezers. Functional spinal units (FSU), each made up of a vertebral-disc-vertebral, were prepared by making axial cuts through the middle of each vertebral body from T12 to L6 with a band saw. The posterior elements were also removed with cuts through both pedicles. Care was taken to ensure that the intervertebral disc of each FSU were intact. FSU were sealed individually in air tight plastic bags and placed in a 2° C. refrigerator for 48 hours to thaw.

Injury to the posterolateral region of each intervertebral disc was created with a stab wound at mid height of the disc using a #11 surgical blade. The surgical blade was used to create a horizontal slit about 8 mm wide and 20 mm deep.

A syringe was used to inject the prepared repair mixture between the slit. The syringe was moved in a retrograde zigzag pattern during injection so as to ensure a good fill into the total area of the slit. Thereafter, the FSU was compressed manually for 2 minutes to assist in better adhesion. Each specimen was again sealed individually in air tight plastic bags and they were placed in a 2° C. refrigerator for 5 days to ensure that all repairs totally cured. Each specimen was subjected to a static intradiscal pressurization test up to 3.5 MPa to determine the maximum pressure that could be sustained without leakage. The repair site was inspected visually during tests for leakage. A water filled needle instrumented with a pressure gauge was inserted anteriorly into the nucleus of the intervertebral disc. The needle was connected to a hydraulic piston and a press was used to compress the hydraulic piston so as to inject the water into the nucleus and to induce a rise in intradiscal pressure.

The results of the experiments were as follows. For Experiment 1 with Ø(PIB-CA)₃ (1.4 g) and Bu-CA (0.6 g), the two materials were mixed in a silylated glass vial under N₂ for 10 minutes. The emulsion was transferred to a disposable polypropylene syringe equipped with an 18 gauge needle. The mixture was injected into the incision in the annulus fibrosis. When the repair site was tested with increased intradiscal pressure an immediate leak was noted at the incision site.

In Experiment 2, conducted with Ø(PIB-CA)₃ (0.8 g) and Oct-CA (0.8 g), the starting materials were mixed in a silylated glass vial under N₂ for 10 minutes. The Oct-CA/Ø(PIB-CA)₃ mixture was transferred to the large cylinder of a double barrel polypropylene syringe (barrel ratio 1:4) equipped with a static mixer. The small barrel of the syringe was filled with Et₂N-PIB-NEt₂ (0.4 g). The Oct-CA/Ø(PIB-CA)₃/Et₂N-PIB-NEt₂ mixture was injected into the incision in the annulus fibrosis. When the repair site was tested with increased intradiscal pressure an immediate leak was noted at the incision site.

In Experiment 3, conducted with Ø(PIB-CA)₃ (0.8 g) and Oct-CA (0.8 g), these starting materials were mixed in a silylated glass vial under N₂ for 10 minutes. The homogeneous mixture was transferred to the large cylinder of a double barrel polypropylene syringe (barrel ratio 1:4) equipped with a static mixer. The small barrel of the syringe was filled with HO-PIB-OH (0.4 g). The Oct-CA/Ø(PIB-CA)₃/HO-PIB-OH mixture was injected into the incision in the annulus fibrosis of 2 specimens. When the repair site was tested with increased intradiscal pressure up to 3.5 MPa, no leak was observed.

In Experiment 4, a comparison was made between the conetwork of Oct-CA/Ø(PIB-CA)₃ and poly(Octyl-CA) (Dermabond®). To begin, Ø(PIB-CA)₃ (1.2 g) and Oct-CA (0.8 g) were mixed in a silylated glass vial under N₂ for 10 minutes. The emulsion was transferred to a disposable polypropylene syringe equipped with an 18 gauge needle. Twelve specimens were repaired with the Oct-CA/Ø(PIB-CA)₃ mixtures and 6 were repaired with Dermabond®.

Each specimen was subjected to static intradiscal pressurization tests first up to 1.7 MPa and second up to 3.5 MPa, followed by a cyclic intradiscal pressurization test for 20 cycles to determine the maximum pressure and number of cycles that could be sustained without leakage. The repair site was inspected visually during tests for leakage. Physiologic intradiscal pressure is up to 2 MPa and thus the applied pressure was higher than the sustained level in normal daily activities. In the static intradiscal pressurization tests, a static intradiscal pressure of 1.7 MPa (250 psi) was reached and next a static intradiscal pressure of 3.5 MPa (500 psi) was maintained for 10 seconds. If leakage occurred below 3.5 MPa, the minimum pressure that would cause leakage was recorded. If no leakage was noted, the specimen was subjected to a cyclic pressurization test. In this final test, 20 cycles of intradiscal pressures ranging between 2. IMPa (300 psi) and 3.5 MPa (500 psi) were applied at a rate of about 1 Hz through the press. If leakage occurred, the number of cycles sustained was recorded.

Further tests were carried out to induce leakage through the repair site of specimens that have not leaked thus far. An 18 gauge needle trocar was introduced through the repair site 5 times. The specimen was then subjected to both static and cyclic intradiscal pressurization as described above. If there was no observable failure, a stab wound was recreated through the repair site with a #11 blade and static pressurization was carried out.

Of the12 specimens repaired with Oct-CA/Ø(PIB-CA)₃, 9 specimens were able to withstand 3.5 MPa for 10 seconds without any visible leakage. Two of the specimens leaked at 0 MPa while 1 leaked at 1.7 MPa (250 psi). In contrast, only 1 of the 6 specimens repaired with octyl-cyanoacrylate was able to withstand 3.5 MPa for 10 seconds without leakage. The other 5 specimens failed at or before 3.5 MPa (1 at I.OMPa, 2 at 1.7 MPa, 1 at 2.4 MPa & 1 at 3.5 MPa). Seven of the 9 remaining specimens repaired with PM-CA survived the 20 cycles of pressurization between 2. IMPa and 3.5 MPa. One of the specimens leaked at the 5th cycle while the other leaked at the 20th cycle. The 1 specimen repaired with octyl cyanoacrylate also survived the 20 cycles of pressurization.

In addition to these tests, the elongation of skin with various wound closure materials or glues applied to the surface of the skin, compared to a control group without any glues applied, was determined. The percent elongation at 10N of applied tensile load of various specimens and a control was compared.

To conduct this test, abdominal porcine skin was procured from a local supplier. The skin was thawed at room temperature and was sectioned into 50 mm by 20 mm strips. These strips of skin specimens were randomized to 4 groups with 6 samples in each group.

Three groups of 6 specimens were randomly applied with poly(ethyl-cyanoacrylate), poly(octyl-cyanoacrylate) and the polymer of the present invention with 20:1 ratio of poly(octyl-CA):Ø(PIB-CA)₃ while the fourth group was not glued. Approximately 0.1 ml of glue was applied over the central 20 mm by 20 mm surface of each specimen. Specimens were stored in an environmental chamber at 80-90% humidity and 2° C. for 48 hours.

Tensile tests were carried out at a rate of 1 mm/second using a universal testing machine. A pair of custom-built specimen clamps was used to firmly grip onto the specimens with a gauge length of 16 mm during tests. Applied displacement and resultant tensile load were recorded at 10 kHz. Subsequently, percent elongation under a 10N tensile load was determined from load-displacement data.

As expected, the control specimens exhibited the best performance, with 57% elongation (standard deviation=13%) under an applied load of 10N (TABLE 2). Specimens applied with poly(ethyl-CA) were much less compliant and exhibited only 16% elongation (s.d.=10%) while specimens applied with poly(octyl-CA) showed 31% elongation (s.d.=5%). Specimens applied with 20:1 Octyl-CA to Ø(PIB-CA)₃ were more compliant and exhibited 54% elongation (s.d.=10%) under the same conditions, as shown in TABLE 2.

TABLE 2
Percent Elongation Under a Tensile Load

| | | | | 20:1 Octyl-CA & |
|---|---|---|---|---|
| | | Ethyl-CA on | Octyl-CA on | PIB-CA on |
| | Pig Skin | Pig Skin | Pig Skin | Pig Skin |
| 1 | 81% | 14% | 31% | 59% |
| 2 | 54% | 11% | 38% | 52% |
| 3 | 56% | 34% | 29% | 45% |
| 4 | 51% | 11% | 36% | 46% |
| 5 | 45% | 11% | 25% | 52% |
| 6 | 57% | 17% | 29% | 72% |
| Mean | 57% | 16% | 31% | 54% |
| Std. Dev. | 13% | 10% | 5% | 10% |

It will be appreciated that a drastic difference in the gradient for elongation compliance between the application site where glue was applied to the skin and the surrounding skin would cause discomfort to the patient and could lead to earlier adhesive failure. According to these experiments, the polymer conetwork was 40% more compliant than poly(octyl-CA), 67% more compliant than poly(ethyl-CA) and it was not significantly different from the control. Thus, the 20:1 Octyl-CA to Ø(PIB-CA)₃ conetwork is superior to both poly(octyl-CA) and poly(ethyl-CA) with compliance similar to that of skin.

Still further, an experiment was carried our to determine the mechanism of wound closure failure and magnitude of wound closure strength when various wound closure materials or glues were used to help secure the apposition of soft tissue.

Abdominal porcine skin was again procured from a local supplier. The skin was thawed in room temperature and was sectioned into 200 mm by 25 mm strips. These strips of skin specimens were randomized into 4 groups with 8 samples in each group.

Each strip of specimen was cut at its mid length and an assigned glue was used to adhere the 2 pieces back together. The 4 groups of specimens were randomly assigned to these 4 glues: 1) Poly(ethyl-CA), 2) Poly(octyl-CA), 3) the copolymer of the present invention with a 20:1 ratio of Octyl-CA:Ø(PIB-CA)₃, and 4) the copolymer of the present invention with a 10:1 ration of Octyl-CA: Ø(PIB-CA)₃ . The glues were applied over the surface of the skin with a width of 5 mm on each of the 2 opposing ends. About 0.05 ml of glue was applied on each specimen. All specimens were kept in an environmental chamber at 80-90% humidity and 2° C. for at least 24 hours.

Tensile tests were carried out at a rate of 1 mm/second using a universal testing machine. Applied displacement and resultant tensile load were recorded at 10 kHz. A pair of custom-built specimen clamps was used to firmly grip onto the specimen during test. The maximum tensile strength and mechanism of failure (glue-skin adhesive failure or glue-glue cohesive failure) were documented.

The mechanism of failure was between the glue and skin (adhesive failure) for the poly(ethyl-CA) group and was between the glue to glue (cohesive failure) for the other 3 glues. The mean failure strength for poly(ethyl-CA) was 8.6N (standard deviation=2.3N), for poly(octyl-CA) was 14.5N (s.d.=3.2N), for 20:1 Octyl-CA:Ø(PIB-CA)₃ was 10.7N (s.d.=2.4N) and for 10:1 Octyl-CA: Ø(PIB-CA)₃ was 9.4N (s.d.=1.6N). A complete listing of the results can be found in TABLE 3.

TABLE 3
Maximum Strength at Failure

| | | 20:1 | 10:1 | |
|---|---|---|---|---|
| | Polyoctyl- | Octyl-CA | Octyl-CA & | Polyethyl- |
| | CA | & PIB-CA | PIB-CA | CA |
| 1 | 18.8 | 7.9 | 12.0 | 5.2 |
| 2 | 18.4 | 11.2 | 8.5 | 6.4 |
| 3 | 12.7 | 9.5 | 8.4 | 9.3 |
| 4 | 10.4 | 11.0 | 9.1 | 7.6 |
| 5 | 13.0 | 12.4 | 9.1 | 10.8 |
| 6 | 10.9 | 11.3 | 10.2 | 11.1 |
| 7 | 16.5 | 14.8 | 10.7 | 8.9 |
| 8 | 15.1 | 7.6 | 6.8 | 9.5 |
| Mean | 14.5 | 10.7 | 9.4 | 8.6 |
| Standard | 3.2 | 2.4 | 1.6 | 2.1 |
| Deviation | | | | |

Wound, closure strength was significantly higher with poly(octyl-CA) over the other 3 glues. However, the main purpose of wound closure glues are to seal wounds and prevent infiltration of bacteria into the wound. The normal physiological elongation of skin is approximately 30% with about 5N of tensile load. Thus, all 4 glues tested would be able to withstand normal physiological loads without catastrophic failure. In a large wound, subdermal sutures would provide closure strength. Accordingly, although poly(octyl-CA) had significantly higher wound closure strength than poly(ethyl-CA) and the polymers tested, both polymers are capable of providing sufficient wound closure strength to withstand physiological loads.

In light of the foregoing, a novel polymer conetwork of poly(octyl cyanoacrylate) and three-arm polyisobutylene stars has been synthesized for use a number of biomedical uses. The polymer conetworks of the present invention exhibit higher elongation than a homopolymer of Oct-CA. Further, the polymer conetworks of the present invention exhibits a strength that is sufficient to maintain two pieces of skin together, which calculated strength is in excess of 5N.

Although the present invention has been described in considerable detail with reference to certain embodiments, other embodiments are possible. Therefore, the spirit and scope of the appended claims should not be limited to the description of the embodiments contained herein.

What is claimed is:

1. A polymer conetwork formed from the polymerization reaction of:
octyl-cyanoacrylate and a tri-telechelic star polymer comprising polyisobutylene terminated with cyanoacrylate groups (Ø(PIB-CA)₃), wherein the ratio of octyl cyanoacrylate to Ø(PIB-CA)₃ is from about 10:1 to about 40:1.

2. The polymer conetwork according to claim 1, wherein polymerization reaction is initiated by nucleophlic groups located on the surface to be covered by the polymer conetwork.

3. The polymer conetwork according to claim 2, wherein the surface to be covered is skin.

4. The polymer conetwork according to any of claims 1 to 3, wherein the polymer conetwork exhibits higher elongation than a homopolymer of octyl cyanoacrylate.

5. The polymer conetwork according to any of claims 1 to 4, wherein the polymer conetwork exhibits a strength of at least 5N and sufficient to maintain two pieces of skin together.

6. Use of the polymer conetwork of claim 1 for wound closure.

7. Use of the polymer conetwork of claim 1 for sealing surgical cuts and puncture wounds.

8. Use of polymer conetwork of claim 1 as an orthopedic sealant.

9. Use of polymer conetwork of claim 1 for repair of torn annulus.
